(19) United States
(12) Patent Application Publication
(10) Pub. No.: US 2021/0404921 A1
(43) Pub. Date: Dec. 30, 2021

(54) PSORIASIS AND OTHER AUTOIMMUNE DISEASES ANTIGEN IMMUNE MODULATOR (AIM) THERAPEUTIC PLATFORM

(71) Applicant: Phaim Pharma Ltd, London (GB)

(72) Inventors: Tihamer Orban, London (GB); Jalahej Heyman, London (GB); Nara Daubeney, London (GB); Piers Daubeney, London (GB)

(73) Assignee: Phaim Pharma Ltd, LONDON (GB)

(21) Appl. No.: 17/358,760

(22) Filed: Jun. 25, 2021

Related U.S. Application Data
(60) Provisional application No. 63/044,586, filed on Jun. 26, 2020.

(51) Int. Cl. G01N 1/40; G01N 1/28; G01N 1/42; C12N 5/0783; G01N 33/50; A61K 35/17; A61P 17/06; A61P 37/06

(52) U.S. Cl. CPC G01N 1/4077; G01N 1/286; G01N 1/42; C12N 5/0636; G01N 33/5091; G01N 2800/56; A61K 35/17; A61P 17/06; A61P 37/06; G01N 2001/2873; G01N 2800/24; G01N 33/505

(57) ABSTRACT

Provided are methods of preparing an immune cell sample from a subject having an autoimmune disorder, the method comprising: obtaining a tissue sample from the subject; and isolating a single immune cell in situ from the tissue sample using laser capture microdissection.

### CROSS-REFERENCE TO RELATED APPLICATIONS

This application claims the benefit of priority to U.S. Provisional Application No. 63/044,586, filed on Jun. 26, 2020. The entire contents of the foregoing application are herein incorporated by reference.

### BACKGROUND

Autoimmune diseases are a global problem. For examples, psoriasis is a chronic autoimmune disease primarily affecting the skin and often joints; the worldwide prevalence is estimated at 2-3%, i.e. about 125 million people. Moreover, the incidence of psoriasis is increasing: in a retrospective cohort of adults, between 1970-1974 the incidence of psoriasis was about 50.8 cases per 100,000 people, and between 1995-1999 the incidence was about 100.5 cases per 100,000 people. Despite its prevalence, the identities of auto-aggressive immune cells and putative targeted self-antigen(s) useful for identification and/or treatment of psoriasis remain to be discovered.

### SUMMARY

Provided are methods of preparing an immune cell sample from a subject having psoriasis, the methods comprising: obtaining a psoriatic tissue sample; and isolating a single immune cell in situ from the psoriatic tissue sample using laser capture microdissection. Provided are methods of preparing an immune cell sample from a subject having psoriasis, the methods comprising: obtaining a psoriatic tissue sample; and isolating a single immune cell in situ from the psoriatic tissue sample using a technique that does not substantially alter the cellular biochemical profile of the immune cell. The isolated single immune cell can be an unmanipulated single immune cell. The technique may comprise one or more of laser capture microdissection (e.g., immunoguided laser capture microdissection), micromanipulation, vacuum pulse assisted technology, immunomagnetic cell separation, density gradient centrifugation, sedimentation, adhesion, aptamers, buoyance-activated cell sorting, fluorescence-activated cell sorting (FACS), and microfluidics.

In some aspects, the psoriatic tissue sample is from about 5 μm to about 15 μm thick. In some aspects, the psoriatic tissue sample is obtained by horizontally cutting a biopsy specimen from a psoriatic lesion. In some aspects, the psoriatic tissue sample is obtained by blending a biopsy specimen obtained from a psoriatic lesion in a micro blender.

Some aspects comprise: (a) compressing the psoriatic tissue sample, and (b) smearing the compressed sample on a glass slide. In some aspects, the compressing is performed with a flat round solid glass head.

In some aspects, the psoriatic tissue sample is obtained by vertically cutting a biopsy specimen obtained from a psoriatic lesion.

Some aspects comprise: (a) freezing the psoriatic tissue sample and (b) sectioning the frozen sample. In some aspects, the freezing comprises laying the specimen on dry ice. In some aspects, the freezing is with liquid nitrogen vapor or isopentane.

Some aspects comprise embedding the tissue sample in an embedding substrate before sectioning the sample. In some aspects, the embedding is performed before freezing the sample. In some aspects, the embedding substrate is tragacanth or an optimal cutting temperature (OTC) medium. In some aspects, the sectioning is performed with a microtome or a cryostat.

In some aspects, the psoriatic tissue sample is obtained by cutting a biopsy specimen obtained from a psoriatic lesion, and wherein the method further comprises: (a) flash freezing the sample, (b) smashing the frozen sample into fragments, (c) thawing the fragments, (d) smashing one or more of the thawed fragments vertically and pushing the thawed fragment horizontally, and then (e) smearing the fragment on a glass slide.

Some aspects comprise culturing the isolated immune cell in a growth medium.

Some aspects comprise detecting T cell surface markers, transcription factors, cytokines, or combinations thereof associated with the isolated immune cell. Some aspects comprise detecting T cell surface markers, transcription factors, cytokines, or combinations thereof associated with one or more of the cultured immune cells. In some aspects, the detecting is performed via flow cytometry, deep sequencing, spectrophotometry, or an enzyme-linked immunosorbent assay.

Some aspects further comprise performing an antigen challenge on the cultured immune cells. In some aspects, the antigen challenge comprises contacting the immune cells with an antigen and then detecting T cell surface markers, transcription factors, cytokines, or combinations thereof associated with one or more of the cultured immune cells or the isolated immune cell (e.g., the unmanipulated single immune cell). In some aspects, the antigen is keratin, melanocytes-ADAMTSL5, or antimicrobial peptide LL37. In some aspects, the antigen is isolated from a skin tissue sample obtained from the subject.

Some aspects comprise (a) homogenizing and fractionating the skin tissue sample, (b) conducting the antigen challenge on the homogenized and fractionated skin tissue sample, (c) detecting fractions that induce T cell stimulation based on the presence or absence of the T cell surface markers, transcription factors, cytokines, newly synthesized DNA, ATP, or combinations thereof, (d) isolating polypeptides in the detecting fractions by performing 3-dimensional gel chromatography, (e) conducting an antigen challenge on the isolated polypeptides, and (f) detecting the isolated polypeptides that induce T cell stimulation based on the presence or absence of the T cell surface markers, transcription factors, cytokines, newly synthesized DNA, ATP, or combinations thereof.

Some aspects comprise detecting, in a peripheral blood sample obtained from the subject, the presence or absence of (i) one or more immune cells having the detected T cell surface markers and/or transcription factors and/or (ii) the isolated polypeptide that induces T cell stimulation.

Some aspects comprise synthesizing one or more of the polypeptides that induce T cell stimulation. Some aspects comprise administering one or more of the polypeptides to a subject having psoriasis.

Also provided are methods of preparing an immune cell sample from a subject having an autoimmune disorder, the method comprising: obtaining a tissue sample from the subject; and isolating a single immune cell in situ from the tissue sample using laser capture microdissection or vacuum pulse assisted technology. Provided are methods of preparing an immune cell sample from a subject having psoriasis, the methods comprising: obtaining a psoriatic tissue sample; and isolating a single immune cell in situ from the psoriatic tissue sample using a technique that does not substantially alter the cellular biochemical profile of the immune cell. The isolated single immune cell can be an unmanipulated single immune cell. The technique may comprise one or more of laser capture microdissection (e.g., immunoguided laser capture microdissection), micromanipulation, vacuum pulse assisted technology, immunomagnetic cell separation, density gradient centrifugation, sedimentation, adhesion, aptamers, buoyance-activated cell sorting, fluorescence-activated cell sorting (FACS), and microfluidics.

In some aspects, the autoimmune disorder is psoriasis, type 1 diabetes mellitus, rheumatoid arthritis, or multiple sclerosis.

In some aspects, the tissue sample is from about 5 μm to about 15 μm thick. In some aspects, the tissue sample is obtained by horizontally cutting a biopsy specimen from an affected tissue. In some aspects the tissue sample is obtained by blending a biopsy specimen obtained from an affected tissue in a micro blender. Some aspects further comprise (a) compressing the tissue sample, and (b) smearing the compressed sample on a glass slide. In some aspects, the compressing is performed with a flat round solid glass head. In some aspects, the tissue sample is obtained by vertically cutting a biopsy specimen obtained from an affected tissue.

Some aspects further comprise: (a) freezing the tissue sample and (b) sectioning the frozen sample. In some aspects, the freezing comprises laying the specimen on dry ice. In some aspects, the freezing is with liquid nitrogen vapor or isopentane. Some aspects further comprise embedding the tissue sample in an embedding substrate before sectioning the sample. In some aspects, the embedding is performed before freezing the sample. In some aspects, the embedding substrate is tragacanth or an optimal cutting temperature (OTC) medium. In some aspects, the sectioning is performed with a microtome or a cryostat.

In some aspects, the tissue sample is obtained by cutting a biopsy specimen obtained from an affected tissue, and wherein the method further comprises: (a) flash freezing the tissue sample, (b) smashing the frozen sample into fragments, (c) thawing the fragments, (d) smashing one or more of the thawed fragments vertically and pushing the one or more thawed fragments horizontally, and then (e) smearing the fragment on a glass slide.

Some aspects further comprise culturing the isolated immune cell in a growth medium.

Some aspects further comprise detecting T cell surface markers, transcription factors, cytokines, newly synthesized DNA, ATP, or combinations thereof associated with the isolated immune cell. Some aspects further comprise detecting T cell surface markers, transcription factors, cytokines, or combinations thereof associated with one or more of the cultured immune cells. In some aspects, the detecting is performed via flow cytometry, deep sequencing, spectrophotometry, or an enzyme-linked immunosorbent assay.

Some aspects further comprise identifying a primary antigen that stimulates the isolated immune cell. In some aspects, the identifying comprises culturing the isolated immune cell to generate cultured immune cells, and performing an antigen challenge the cultured immune cells. In some aspects, the antigen challenge comprises contacting the immune cells with an antigen and then detecting T cell surface markers, transcription factors, cytokines, or combinations thereof associated with one or more of the cultured immune cells or the isolated immune cell (e.g., the unmanipulated single immune cell). In some aspects, the antigen is isolated from a tissue sample obtained from the subject.

Some aspects further comprise: (a) homogenizing and fractionating the tissue sample, (b) conducting the antigen challenge on the homogenized and fractionated tissue sample, (c) detecting fractions that induce T cell stimulation based on the presence or absence of the T cell surface markers, transcription factors, cytokines, newly synthesized DNA, ATP, or combinations thereof, (d) isolating polypeptides in the detected fractions by performing 3-dimensional gel chromatography, (e) conducting an antigen challenge on the isolated polypeptides, and (f) detecting the isolated polypeptides that induce T cell stimulation based on the presence or absence of the T cell surface markers, transcription factors, cytokines, or combinations thereof.

Some aspects further comprise detecting, in a peripheral blood sample obtained from the subject, the presence or absence of (i) one or more immune cells having the detected T cell surface markers and/or transcription factors and/or (ii) the isolated polypeptide that induces T cell stimulation.

Some aspects further comprise synthesizing one or more of the polypeptides that induce T cell stimulation.

Also provided are methods of treating a subject having an autoimmune disorder, the method comprising administering to the subject a therapeutically effective amount of the identified primary antigen/s.

Some aspects further comprise culturing the immune cell in a growth medium that induces formation of regulatory immune cells. Some aspects comprise methods of treating a subject having an autoimmune disorder, the method comprising administering to the subject a therapeutically effective amount of the induced regulatory immune cells.

### DETAILED DESCRIPTION

As used herein, the singular forms “a”, “an”, and “the” include both singular and plural referents unless the context clearly dictates otherwise.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, such as variations of +/−10% or less from the specified value, unless specifically identified to mean a separate variation, such as +1-5% or less, +/−1% or less, or +/−0.1% or less of and from the specified value. It is to be understood that the value to which the modifier “about” refers is itself also specifically, and preferably, disclosed.

The terms “quantity”, “amount” and “level” are synonymous and generally well-understood in the art. The terms may particularly refer to an absolute quantification of a marker in a tested object (e.g., in or on a cell, cell population, tissue, or organ, e.g., in a biological sample of a subject), or to a relative quantification of a marker in a tested object, i.e., relative to another value such as relative to a reference value, or to a range of values indicating a base-line of the marker. For example, the base-line or reference value can be obtained based on a determination of the quantity, level, or amount of a marker in a subject (or in cells or populations of cells from the subject) having an autoimmune disorder or not having an autoimmune disorder or one or more symptoms thereof. Such values or ranges may be obtained as conventionally known. In some cases, the quantity, amount, or level is a measured concentration. Amounts can be quantified using known techniques, such as PCR, UV absorption, calorimetry, fluorescence-based measurement, diphenylamine reaction methods, and others.

Described are unique immune modulatory platforms to diagnose and/or treat autoimmune diseases, such as psoriasis. Without being bound by theory, it is believed that autoimmune disorders, including psoriasis, are caused by the loss of self-tolerance to a particular self-antigen. The self-antigen triggers an autoimmune process that targets the organ to which that particular self-antigen is specific. For instance, in type 1 diabetes mellitus the self-antigen is in the insulin producing pancreatic beta cells; in psoriasis the self-antigen is in the skin; in autoimmune thyroiditis, the antigen is in the thyroid gland, etc.

Psoriasis is an autoimmune disease that has several phenotypes, including plaque (also known as psoriasis vulgaris), guttate, inverse, pustular, and erythrodermic. Psoriasis has strong genetic components located in the HLA regions. One such locus is psoriasis susceptibility locus 1 (PSORS1). The strongest HLA-related associations in psoriasis map to HLA-C*06 in the white population. The disease is characterized with remissions and flare ups. Prevalence of psoriasis among the first-degree relatives is high, i.e., about 4-19% higher than for the general population.

Psoriasis pathophysiology is characterised by abnormal keratocyte proliferation and immune cell infiltration in the epidermis and dermis. Without being bound by theory, it is believed that the abnormal recognition and presentation of putative autoantigen(s) triggers auto-aggressive T cell populations, including Th1 and Th17 cells. The disease can be maintained and exacerbated by pro-inflammatory cytokines (e.g., TNF-alpha, IL-17, IL-22 and 23, CCL20). Epithelial damage can trigger anti-microbial peptides such as CAMP which then mediates plasmacytoid dendritic cell (pDC) activation further fuelling the skin destruction.

Described are methods of recapitulating the process by which healthy individuals acquire self-tolerance to the body's immune system. For instance, in healthy individuals, immune cells are circulated in the baby's thymus early in life where self-antigens of different organs are expressed; some immune cells (which are specific to a particular antigen(s)) get eliminated; some immune cells are not eliminated, but they are kept under control by regulatory immune cells also specific to these self-antigens. Autoimmunity can occur when these regulatory cells lose control over the immune cells that are not eliminated. Thus, some aspects comprise introducing a self-antigen to a subject to stimulate regulatory cells to regain control and re-establish the healthy immune balance.

An “antigen” is a structural substance, such as a protein or polypeptide, that is recognized by the immune system and serves as a target for an immune response. A “self-antigen” or “autoantigen” is an antigen derived from an organism which under normal circumstances is not recognized by the immune system of that organism, but which may become a target of immune attack, resulting in an autoimmune disease.

Against the above backdrop, provided are methods and compositions for identifying T cells and/or antigens in a subject having an autoimmune disorder. In some aspects, the subject has been diagnosed with an autoimmune disorder. In some aspects, the subject is at risk of developing an autoimmune disorder. In some aspects, the autoimmune disorder is psoriasis. In some aspects, the autoimmune disorder is rheumatoid arthritis. In some aspects, the autoimmune disorder is type 1 diabetes mellitus. In some aspects, the autoimmune disorder is multiple sclerosis.

Also provided are methods and compositions for treating or preventing an autoimmune disorder in a subject.

### Methods of Preparing a Tissue Sample

Provided are novel and inventive methods of preparing immune cell samples from a subject having an autoimmune disorder, such as psoriasis. Such methods can be used, for instance, to identify immune cells present in the tissue of a subject.

Some aspects comprise obtaining a tissue sample from a subject. The tissue can be any tissue impacted by an autoimmune condition, such as the skin, pancreatic tissue, spinal fluid, synovial fluid, etc. For instance, the tissue for a subject having psoriasis can be the skin. Exemplary psoriatic tissue includes psoriatic plaques or lesions, red and scaly skin with pustules, red spots, bright red shiny lesions, and/or scales. The tissue for a subject having multiple sclerosis can be cerebrospinal fluid (e.g., obtained from a spinal fluid tap). The tissue from a subject having type 1 diabetes mellitus can have pancreatic cells. The tissue from a subject having rheumatoid arthritis can be synovial fluid.

In some aspects, the tissue sample is obtained during an autoimmune outbreak (e.g., a psoriatic flare-up). In some aspects, the tissue sample is obtained during an autoimmune condition (e.g., psoriasis) remission or partial remission. In some aspects, tissue sample is isolated from serial biopsies obtained during the course of the condition, e.g., to provide information related to dynamic changes in the autoimmune process and its self-regulatory healing process.

The tissue sample can be obtained by any of a variety of methods. For instance, the tissue sample can be obtained directly from a subject or from a biopsy specimen obtained from a subject. Exemplary types of biopsies include a shave biopsy, a punch biopsy, an incisional biopsy, and an excisional biopsy. In some aspects, the biopsy is obtained by removing, from a subject, some or all of an impacted tissue with a scalpel. In some aspects, individual cells (e.g., from cerebrospinal fluid or synovial fluid) can be picked up from solution, e.g., using a microscope with a micromanipulator.

In some aspects, the tissue sample is obtained by horizontally cutting (e.g., parallel to the skin layers) a biopsy specimen obtained from a subject's tissue (e.g., a psoriatic lesion). In some aspects, the tissue sample is obtained by vertically cutting a biopsy specimen obtained from a subject's tissue. Such cutting can be performed with any known means, such as with a scalpel. In some aspects, the tissue sample is obtained by blending a biopsy sample obtained from a subject's tissue in a micro blender.

In some aspects, the tissue sample obtained from the biopsy specimen is further treated. In some aspects, the further treatment does not include mincing or digesting the tissue sample. In some aspects, the tissue sample can be compressed (flatten tissue in a vertical direction using constant or variable pressure, e.g., manually or using a machine assisted process and a hard surface), smashed (hit or compress such that the tissue separates into smaller and thinner pieces), pushed (transfer the tissue through a defined diameter to separate and/or make it thinner), pulled, smeared, frozen, or any combination thereof.

In some embodiments, the tissue sample is manually or mechanically compressed, e.g., with a flat round solid glass head, or with a mortar and pestle, or with a presser or crusher. For instance, the head can be rotated and pushed downward and sideways to mechanically compress the sample. In some aspects, the compressed sample is smeared on a glass slide, which can then be used for microdissection of the sample.

In some aspects, the tissue sample is frozen, e.g., using flash freezing or snap freezing techniques. Exemplary freezing techniques include exposure to dry ice, liquid nitrogen vapour, or isopentane. In some aspects, the tissue sample is cryoprotected (e.g., by exposing the tissue sample to a cryoprotectant, such as 4% Paraformaldehyde and/or sucrose) prior to being frozen. Preferably, the tissue sample is frozen without ice crystals developing in the cells of the sample.

Some aspects comprise fragmenting the frozen tissue sample. Such fragmentation can be obtained by smashing the frozen tissue sample into fragments, for instance via manual or mechanical compression, or with a micro blender. In some aspects, the fragmented tissue samples are thawed, and then further compressed or pushed (e.g., by smashing the thawed fragment vertically and/or pushing them horizontally). In some aspects, the fragmented tissue samples are smeared on a glass slide.

In some aspects, the tissue sample is embedded in a substrate. The substrate can be a gum or adhesive. Exemplary substrates include pol[N-(2-hydroxypropyl)methacrylamide], 10% gelatin, carboxymethyl cellulose, tragacanth, and combinations thereof. In some aspects, the substrate is in an optimal cutting temperature (OCT) medium. Exemplary OCT media include water-soluble blends of glycols and resins, e.g., that provide a matrix for sectioning the tissue sample. See, e.g., FISHER HEALTHCARE™ TISSUE-PLUS™ O.C.T. Compound, or TISSUE-TEK® O.C.T. Compound (SAKURA® Finetek). In some aspects, a frozen tissue sample is embedded in the substrate. In some aspects, a fresh tissue sample is embedded in the substrate. In some aspects, a fragmented tissue sample is embedded in the substrate.

Some aspects comprise sectioning (e.g., cryosectioning), or slicing, the tissue sample. For instance, the tissue sample can be sectioned using a blade or knife. In some aspects the tissue sample is sectioned using a microtome. In some aspects the tissue sample is sectioned using a cryostat.

The smeared or sectioned tissue samples can be any thickness suitable for microdissection. For instance, in some aspects the smeared or sectioned tissue sample is from about 5 microns to about 15 microns thick, such as about 8-10 microns thick. In some aspects the smeared or sectioned tissue sample is from 5 microns to 15 microns thick, such as 8-10 microns thick. In some aspects, the smeared or sectioned tissue sample has a thickness of about 5 microns, about 6 microns, about 7 microns, about 8 microns, about 9 microns, about 10 microns, about 11 microns, about 12 microns, about 13 microns, about 14 microns, or about 15 microns. In some aspects, the smeared or sectioned tissue sample has a thickness of 5 microns, 6 microns, 7 microns, 8 microns, 9 microns, 10 microns, 11 microns, 12 microns, 13 microns, 14 microns, or 15 microns. In some aspects, the smeared or sectioned tissue sample comprises a monolayer of cells. In some aspects, a portion of the smeared or sectioned tissue sample comprises a monolayer of cells.

In some aspects the smeared or sectioned tissue sample is placed on a slide (e.g. a glass slide) or membrane for microdissection.

### Immune Cell Isolation

Also provided are methods for isolating one or more immune cells from a tissue sample. The term “immune cells” refers to cells of the innate or acquired immune system, and includes, without limitation, lymphocytes, such as T cells and B cells (including any cell in the B cell lineage), naïve cells, memory cells, antigen-presenting cells (APC), dendritic cells, monocytes, macrophages, natural killer (NK) cells, mast cells, basophils, eosinophils, or neutrophils, as well as any progenitors of such cells. In certain preferred aspects, the immune cell may be a T cell. As used herein, the term “T cell” (i.e., T lymphocyte) is intended to include all cells within the T cell lineage, including thymocytes, immature T cells, mature T cells and the like. The term “T cell” may include CD4⁺ and/or CD8⁺ T cells, T helper (Tₕ) cells, e.g., Tₕ1, Tₕ2 and Tₕ17 cells, T regulatory (T_{reg}) cells, NKT-cells (including variant and/or invariant cells), and gamma T cells.

In some aspects, the immune cell is isolated from a tissue obtained during an autoimmune outbreak (e.g., a psoriatic flare-up). In some aspects, the immune cell is isolated from a tissue obtained during an autoimmune condition (e.g., psoriasis) remission or partial remission. In some aspects, immune cells are isolated from serial biopsies obtained during the course of the condition, e.g., to provide information related to dynamic changes in the autoimmune process and its self-regulatory healing process.

In some aspects, the immune cell is isolated from a smeared tissue sample. In some aspects, the immune cell is isolated from a sectioned tissue sample. The smeared or sectioned tissue sample can be obtained in accordance with the methods set forth above.

The immune cell can be isolated using microdissection techniques, such as laser capture microdissection. Laser capture microdissection is described, for instance, in Datta et al., “Laser capture microdissection: Big data from small samples,” Histol Histopathol., 30(11): 1255-1269 (2015), which is herein incorporated by reference in its entirety. Briefly, laser capture microdissection allows for the separation of a cell of interest from a tissue sample using an inverted light microscope (with or without a fluorescent module) fitted with a laser to facilitate the visualization and procurement of cells. Some aspects utilize infrared laser capture microdissection. Some aspects utilize ultraviolet laser capture microdissection. In some specific aspects, the laser capture microdissection is immunoguided laser capture microdissection. Immunoguided laser capture microdissection combines immunostaining with laser capture microdissection, allowing immunophenotypes to be used, in addition to morphology and tissue location, to identify and isolate target cells from the tissue sample. This technique employs immunohistochemistry or immunofluorescence to guide the dissection process for isolating cells expressing a specific molecular marker, and may be particularly useful when histological stains do not recognize certain cell populations.

After the cell of interest is separated from the adjacent tissue, it can be extracted from the tissue sample. In some aspects, the cell of interest is transferred from a slide using a non-contact method, such as with gravity (e.g., gravity-assisted microdissection), pressure catapulting (e.g., laser pressure catapult), or laser induced forward transfer. In some aspects, the cell of interest is transferred from a slide by pressing a sticky surface to the cell and tearing it off the slide. In some aspects, the cell is extracted using a heat transfer process that adheres the cell of interest to a membrane without adhering adjacent tissues. In some aspects, the cell is extracted using a vacuum pulse assisted technology which may allow for manual or automatic single cell isolation, for example using microdissection instruments such as UnipicK™, UnipicK+™ and A-picK™ (NeuroInDx, Inc.™).

In some aspects, the immune cell can be isolated by micromanipulation. Micromanipulation, a form of manual cell picking, is a cell isolation technique involving the use of an inverted microscope and ultra-thin glass capillaries connected to an aspiration and release unit. The system moves through motorized mechanical stages, allowing the operator to carefully select a specific cell and apply suction via micropipette to aspirate and isolate the cell. In specific aspects, micromanipulation may be performed with vacuum pulse assisted technology, which may allow for manual or automatic single cell isolation, for example using microdissection instruments such as UnipicK™, UnipicK+™ and A-picK™ (NeuroInDx, Inc.™).

In some aspects, the immune cell can be isolated using immunomagnetic cell separation. Immunomagnetic cell separation is a technique whereby magnetic particles are used to isolate target cells from heterogeneous mixtures. To accomplish this, the magnetic particles are bound to specific cell surface proteins on the target cells via antibodies, enzymes, lectins, or streptavidin. The sample is then placed in an electromagnetic field that pulls on the magnetic particles, bringing the labeled cells with them. The unlabeled cells remain in the tissue sample.

In some aspects, the immune cell can be isolated using density gradient centrifugation (e.g., in fluid tissue samples). Density gradient centrifugation relies on the varying densities of cells within a heterogeneous sample. The sample is layered on top of a density gradient medium before being centrifuged. During centrifugation, each cell type will sediment to its isopycnic point, which is the place in the medium gradient where the density of the cells and medium are equal.

In some aspects, the immune cell can be isolated using sedimentation. Sedimentation works on the basis that gravity will cause larger and denser components to sediment faster than materials that are smaller and less dense. The largest and densest components in a sample can be pelleted through an initial low-force centrifugation due to their high rate of sedimentation. The supernatant can then be spun again. Through successive centrifugations, components with an increasingly lower rate of sedimentation can be isolated. Leukocytes are commonly separated from erythrocytes through dextran sedimentation.

In some aspects, the immune cell can be isolated using adhesion. The unique adhesion profiles of different cell types can be used to separate target cells in a fluid sample from heterogeneous populations. By choosing suitable growth factors and cell culture plates to selectively favor or inhibit adhesion, adherent cells can be separated from cells in suspension. For example, macrophages are inherently adherent and often isolated from peripheral blood and bone marrow by adhesion. Mononuclear cells can be cultured with serum and a differentiation cocktail, promoting the formation of an adherent monolayer of macrophages. After removing the supernatant containing unwanted cells, the macrophages can be isolated. Alternatively, cells that naturally grow in suspension or have lost anchorage dependency can be isolated by culturing the heterogeneous cell population in plates designed for ultra-low attachment. Without a surface to adhere to, adherent cells will fail to survive and the target cells will remain in suspension.

In some aspects, the immune cell can be isolated using aptamers. Aptamers are single-stranded RNA or DNA oligonucleotides that form structures that can bind to highly specific targets. Through systematic evolution of ligands by exponential enrichment (SELEX) technology, aptamers can be screened and synthesized to target any cell type. Systematic Evolution of Ligands by EXponential enrichment (SELEX) is an experimental procedure that allows extraction, from an initially random pool of oligonucleotides, of the oligomers with a desired binding affinity for a given molecular target. These aptamers have high affinity and specificity toward their targets, and can be labeled with fluorochromes or magnetic particles to facilitate cell separation. Aptamers lack immunogenicity.

In some aspects, the immune cell can be isolated by buoyance-activated cell sorting. Buoyancy-Activated cell sorting is a cell separation technique that typically utilizes glass microbubbles labeled with antibodies specific to the target cells. When mixed into the sample, the microbubbles bind to the target cells. Due to the augmented buoyancy force, the microbubbles float to the surface, separating the target cells.

In some aspects, the immune cell can be isolated by fluorescence-activated cell sorting (FACS). FACS is a method that uses flow cytometry and fluorescent probes to sort heterogeneous mixtures of cells. Fluorophore-tagged antibodies bind to epitopes on specific antigens on the target cells within a single-cell suspension. After tagging, the flow cytometer focuses the cell suspension into a uniform stream of single cells. This stream is then passed through a set of lasers that excites the cell-bound fluorophores, causing light scattering and fluorescent emissions. Based on the wavelengths produced by the laser excitation, the resulting photon signals are converted into a proportional number of electronic pulses that assign a charge to the droplet that is formed around the cell. As each droplet falls between the deflection plates, its charge causes the droplet to either be deflected into collection tubes or fall into the waste chamber. In some implementations, FACS may be used to isolate single cells from enriched samples of isolated cells, including samples prepared by other means disclosed herein (e.g., immunomagnetic cell separation, density gradient centrifugation, sedimentation, adhesion, etc.).

In some aspects, the immune cell can be isolated using microfluidics. Microfluidics manipulates fluids on a microscopic level to facilitate single-cell isolation. Microfluidic technologies are frequently built onto microchips and are commonly known as “lab-on-a-chip” devices. These devices have several advantages, including the smaller volumes of samples and reagents required for use. Lab-on-a-chip devices are also portable, making them particularly useful as field-based diagnostic tools. Microfluidic methods can be divided into active and passive systems. Active microfluidic systems involve external forces, whereas passive microfluidics make use of the cell's density and mass in combination with gravity. These methods can also be classified by the presence or absence of cell labelling. There are several different microfluidic methods used for cell isolation, including, for example: acoustophoresis, aqueous two phase systems, biomimetic microfluidics, cell affinity chromatography, deterministic lateral displacement, electrophoretic sorting, field flow fractionation, gravity and sedimentation, magnetophoresis, microfiltration, and optical sorting. In some implementations, microfluidics may be used to isolate single cells from enriched samples of isolated cells, including samples prepared by other means disclosed herein (e.g., immunomagnetic cell separation, density gradient centrifugation, sedimentation, adhesion, etc.).

In some aspects, the cell of interest is preferably isolated and extracted from the tissue sample in a manner that preserves the cell viability. In some aspects, the cell of interest is preferably isolated and extracted from the tissue in a manner that does not alter or does not substantially alter the cellular biochemical profile (e.g., the proteome, genome, and/or transcriptome). For example, the cell of interest can be isolated and extracted from the tissue in a manner such that the cell remains unmanipulated (e.g., an unmanipulated single immune cell can be isolated and extracted from the tissue). The manner of isolation and extraction may substantially preserve, for example, the cell surface receptors present on the cell. Some techniques of extracting cells from tissue samples, such as, for example, techniques involving tissue digestion agents and/or embedding agents that do not preserve the cells within the tissue (e.g., paraffin or electron microscopy resin) may prevent the ability to profile individually isolated cells, may alter the cellular biochemical profile of the cells within the tissue sample, and/or may kill the cells, preventing the ability to characterize the in vitro cellular response to stimulation relevant for identifying auto-antigens, as described elsewhere herein. In some embodiments, the immune cell (e.g., T-cell) may be isolated from a blood sample and challenged with an antigen identified from a tissue sample as described elsewhere herein. Performing antigen challenges on immune cells isolated and extracted from a relevant tissue sample (e.g., a tissue that experiences immune cell infiltration during an autoimmune response), however, may be advantageous in providing higher concentrations or proportions/frequencies of immune cells that are specific for the autoantigen of interest.

In some aspects, the cell of interest is cultured in a growth medium, e.g., to produce a colony of cells. In some aspects, the culture medium is a complete medium and effects IL-12 stimulation.

In some aspects, the cell(s) of interest is cultured in a growth medium that induce the formation of T_{reg} cells, or cells having T_{reg} activity. In some aspects, the cell(s) of interest are cultured in a growth medium that induces Foxp3 expression. In some aspects, the cells are cultured in a growth medium that contains one or more of TGF-β, IL-10, IL-4 and IL-35. Some aspects comprise conversion to T_{reg} cells in accordance with the methods set forth in Chen et al., J. Experimental Medicine, 198(12): 1875-1886 (2003); Cao et al., J. Am. Soc. Nephrol., 21: 933-942 (2010); or Collison et al., Nat. Immunol., 11(12): 1093-1101 (2010), the disclosures of each of which are incorporated herein in their entireties.

### Immune Cell Identification

Also provided are methods of characterizing isolated immune cells (e.g., a single immune cell) or a colony of cells cultured in a growth medium. Such characterization can include, but is not limited to analysing the genomics, transcriptomics, proteomics, and/or metabolomics of the immune cell. Some aspects comprise determining expression levels of surface markers, transcription factors, cytokines, signal transducers, or combinations thereof associated with the immune cell. “Expression” refers to the process by which a polynucleotide is transcribed from a DNA template (such as into a mRNA or other RNA transcript) and/or the process by which a transcribed mRNA is subsequently translated into peptides, polypeptides, or proteins. Thus, “expression levels” can refer to an amount of a nucleic acid (e.g. mRNA) or protein in a sample.

In some aspects, the immune cell is a T cell. Exemplary T cell surface markers include T cell receptors (TCRs), CD3, CD4, CD8, CXCR3, CCR4, CD25, CD127, FoxP3, CD19/20, CD196 (CCR6), CD197 (CCR7), CD62L, CD123, CD 80/86, CD69, and CD45RO/RA. Exemplary T cell transcription factors include Aiolos, ATM, BATF, Bcl-6, Blimp-1, FOXP3, GATA3, Helios, Ikaros, IRF4, IRF7, Ki-67, NF-kB p65, p53, PCNA, SSRP1, STAT1, STAT4, T-bet, TCL1, Th-POK, and ZAP-70. Exemplary T cell cytokines include INF-γ, IL-17, TNF-α, IL-6, IL-4, IL10, TGF-β, IL-12, IL-13, IL-2, and IL-23. Exemplary T cell signal transducers include JAK1/3, PI3K, etc.

Surface markers, transcription factors, cytokines, and signal transducers can be identified by any suitable means. For instance, some aspects comprise using flow cytometry to identify the surface markers, transcription factors, and/or cytokines. Some aspects comprise using spectrophotometry. Some aspects comprise using an enzyme-linked immunosorbent assay. Some aspects comprise using deep sequencing. Some aspects comprise performing RNA-Seq or single-cell RNA-Seq. Some aspects comprise performing single-cell genome (DNA) sequencing. Some aspects comprise using tetramer or dextramer technology, e.g., DCODE™ DEXTRAMER®, which can provide insight into antigen specificity, T-cell receptor sequences, and/or genomic profiling.

Some aspects comprise identifying T cell receptors (TCRs) associated with isolated T cells. More particularly, TCR α and/or β chains can be characterized using antibodies and/or RT-PCR.

In some aspects, immune cells from an autoimmune outbreak (e.g., a psoriatic flare-up) are identified. In some aspects, the immune cell is isolated from a tissue obtained during an autoimmune condition (e.g., psoriasis) remission or partial remission. In some aspects, the immune cells are isolated from serial biopsies obtained during the course of the condition, e.g., to provide information related to dynamic changes in the autoimmune process and its self-regulatory healing process. For example, CD25hi+/CD127 low, FoxP3+ population frequency and also the naïve and memory T cell population's ratios can be characterized in the serial biopsies to provide such information related to the dynamic changes in the autoimmune process and its self-regulatory healing process.

### Antigen Challenge and Identification

Also provided are methods of performing an antigen challenge on cultured immune cells. In some aspects, cultured immune cells are contacted with an antigen, and the impact of exposing the immune cells to the antigen is determined, e.g., by measuring expression levels of surface markers, transcription factors, newly synthesized DNA (e.g., as measured by -3H-thymidine incorporation), ATP, cytokines, cell signal transducers, or combinations thereof associated with the immune cell. Antigen challenges are disclosed, for example, in Ridgway et al., “Following Antigen Challenge, T Cells Up-Regulate Cell Surface Expression of CD4 In Vitro and In Vivo,” J. Immunol., 161: 714-720 (1998), and Brinke et al., “Monitoring T-Cell Responses in Translational Studies: Optimization of Dye-Based Proliferation Assay for Evaluation of Antigen-Specific Responses,” Fontiers Immunol., 8: 1870 (2017), both of which are herein incorporated by reference in their entirety. Some aspects comprise predicting antigen specificity of a T cell using in-depth analysis of its TCR, e.g., as set forth in Fischer et al., “Predicting antigen-specificity of single T-cells based on TCR CDR3 regions,” buiRxiv (Aug. 13, 2019), which is herein incorporated by reference in its entirety.

In some aspects (e.g., in the case of psoriasis) the antigen used in the antigen challenge is a keratocyte (e.g., keratin); a melanocyte (e.g., ADAMTSL5); an antimicrobial peptide (e.g., LL37); post-translationally-modified variants of any of the foregoing; fragments of any of the foregoing; and/or any combination thereof. In some aspects (e.g., in the case of multiple sclerosis), the antigen used in the antigen challenge is Myelin basic protein (MBP), Myelin oligodendrocyte glycoprotein (MOG) peptides, alpha-beta-crystallin, S100beta, the DM20 isoform of proteolipid protein (PLP) myelin-associated antigen (MAG), myelin-associated oligo-dendrocyte basic protein (MOBP), 2′,3′-cyclic-nucleotide 3′-phosphodiesterase (CNPase), post-translationally-modified variants of any of the foregoing, fragments of any of the foregoing, and/or any combination thereof. In some aspects (e.g., in the case of type 1 diabetes mellitus), the antigen used in the antigen challenge is preproinsulin, proinsulin, insulin A chain, insulin B chain, insulin C chain, GAD65, ICA512/Ia2, HSP60, carboxypeptidase H, peripherin, ganglioside, post-translationally-modified variants of any of the foregoing, fragments of any of the foregoing, and/or any combination thereof. In some aspects (e.g., in the case of rheumatoid arthritis), the antigen used in the antigen challenge is collagen type II; proteoglycan; human chondrocyte glycoprotein 39; a heat shock protein (HSP) (such as BiP); a post-translationally modified protein (such as citrullinated filaggrin); a ubiquitous protein (such as glucose-6-phosphate isomerase); p205; N-acetylglucosamine-6-sulfatase (GNS); filamin (FLNA); post-translationally-modified variants of any of the foregoing; fragments of any of the foregoing; and/or any combination thereof.

In some aspects, the antigen is isolated from a tissue sample obtained from a subject having an autoimmune condition. Such antigens can be isolated for instance from tissue (e.g., psoriatic tissue) obtained from the subject, e.g., via a biopsy. In some aspects the tissue (e.g., psoriatic tissue) is minced and/or digested. In other aspects, the tissue (e.g., psoriatic tissue) is not minced and/or is not digested.

Some aspects comprise homogenizing the tissue. Some aspects comprise fractionating the tissue. Some aspects comprise conducting an antigen challenge on homogenized and fractionated tissue. Some aspects comprise detecting fractions that induce T cell stimulation in the antigen challenge. Some aspects comprise further fractionating the tissue to identify the smallest-responding fraction. Some aspects comprise isolating polypeptides (e.g., proteins) from one or more fractions that induce T cell stimulation. Such isolation can occur using chromatography techniques, such as 3-dimensional chromatography. Some aspects comprise isolating polypeptides based on properties such as size, charge, and/or hydrophobicity. Some aspects comprise additionally using mass spectrometry methods to isolated the polypeptide, such as those described in Duong et al, “Review of Three-Dimensional Liquid Chromatography Platforms for Bottom-Up Proteomics,” Int'l. J. Molecular Sci., 21(1524): 1-19 (2020), which is herein incorporated by reference in its entirety. Some aspects comprise conducting an antigen challenge on one or more of the isolated polypeptides. Some aspects comprise identifying the isolated polypeptides that induce T cell stimulation.

### Autoimmune Detection/Treatment

Also provided are methods of detecting T cells and/or antigens in a subject having, or suspected of having, and autoimmune disorder (such as psoriasis). Some aspects comprise detecting the T cell and/or antigen in tissue (e.g., skin tissue) obtained from the subject. Some aspects comprise detecting the T cell and/or antigen in a blood sample (e.g., a peripheral blood sample) obtained from the subject. Some aspects comprise identifying a subject having the T cells and/or antigens as having the autoimmune disorder (e.g., psoriasis). Some aspects comprise diagnosing the subject as having the autoimmune disorder based on the presence of the T cells and/or antigens. The antigen may be an autoantigen identified from a tissue sample according to any of the methods described elsewhere herein and/or the T cell may be a T cell having antigen specificity for an autoantigen identified from a tissue sample according to any one of the methods described elsewhere herein. Some aspects comprise determining the stage of the autoimmune disorder (e.g., flare-up, remission, partial remission, etc.) based on the presence of the T cells and/or antigens. Some aspects comprise determining the likelihood the subject will respond favourably to treatment.

Also provided are methods of treating (including prophylactic treatments) a subject identified has having an autoimmune disorder or being at risk of having an autoimmune disorder. Treatment includes any type of measure that imparts a benefit on a patient afflicted with or at risk of developing an autoimmune disorder or symptoms thereof. Such benefits can include slowing, controlling, reversing, or stopping the progression of one or more symptoms of the disorder and/or the disease process itself. Such treatment can include, but does not require, a total elimination of all symptoms or a cure of the disorder.

Some aspects comprise administering to a subject in need thereof a therapeutically effective amount of an antigen/s, such as an antigen/s identified in accordance with the methods described above. In some aspects, administration of the antigen/s provides a beneficial shift toward regulatory immune cells and/or downregulates the pathologic immune response. Some aspects comprise isolating or synthesizing the antigen(s) for administration to the subject. In some aspects, the antigen(s) is administering with an adjuvant(s) and/or pharmaceutically acceptable vehicle(s). In various aspects, the antigen(s) may be administered according to any of the methods or general formulations described in U.S. Pat. App. Pub. No. 2016/0361397 to Orban et al., published Dec. 15, 2016, which is herein incorporated by reference in its entirety.

Some aspects comprise administering, to a subject, T_{reg} cells, or cells having T_{reg} activity. In some aspects the T_{reg} cells, or cells having T_{reg} activity are produced by culturing cell(s) of interest in a growth medium that induces the formation of cells have T_{reg} activity. In some aspects, the administered cells express Foxp3. In some aspects, the administered cells are produced by culturing cell(s) of interest in a growth medium that contains one or more of TGF-β, IL-10, IL-4 and IL-35.

The following examples are included as illustrative of the compositions and methods described herein. The examples are in no way intended to limit the scope of the invention. Other aspects will be apparent to those skilled in the art.

### EXAMPLES

### Example 1: Immune Cell Isolation

Untreated, un-manipulated immune cells are isolated from target organ lesions. Instead of mincing and digesting the skin tissue to isolate immune cells, a laser single cell capturing method—Laser Capture Microdissection (LCM)—is used. Without being bound by theory, it is believed that such techniques allow access to the unaltered immune cells in a solid tissue sample for laser capturing without degradation of the extracellular matrix. The tissue for performing LCM is about 5-15 microns thick, as compared e.g. to skin biopsies which are generally from 6-8 mm thick.

The tissue or unaltered cells are obtained from an autoimmune disease-affected site (e.g., affected tissue, such as joint fluid or biopsy in rheumatoid arthritis; cerebrospinal fluid or brain biopsy in multiple sclerosis; pancreatic biopsy in type 1 diabetes mellitus; skin biopsy in psoriasis). The isolated cells are placed on a glass slide or, alternatively, the tissue is prepared in accordance with any of the below techniques.

SAS method (Smash And Smear): Using sharp scalpel for microdissection, fresh tissue samples are cut from a psoriatic lesion in a horizontal manner (i.e. parallel to the skin layers) as thin as possible (intact part of dermis and fat tissue is discarded). Alternatively, a micro blender can be used to disintegrate the psoriatic lesion into small pieces. Then, the sample is smashed with a flat round solid glass head by rotating the head and pushing it downward and sideways to further mechanically compress the samples on a hard surface (each thin cut sample is smashed separately). Using a sharp edged glass slide, each smashed cut is smeared on a glass slide.

FAST method #1 (Flash-freeze And Slice, Thaw): Using a sharp scalpel for microdissection, fresh tissue samples are cut from a psoriatic lesion in a vertical manner (i.e. across the skin layers) as thin as possible (cut intact tissue i.e. fat etc.). The slices are laid on dry ice, and they can be stored in sterile container at −80° C. or used right away. Without being bound by theory, it is believed that the cells are frozen without ice crystals developing in the cells. The slices are then embedded (e.g., in tragacanth) and a microtome/cryostat is used to slice the specimen to the desired thickness (e.g., 8-10 micron).

FAST method #2 (Flash-freeze And Slice, Thaw): Using a sharp scalpel for microdissection, fresh tissue samples are cut from the psoriatic lesion in a vertical manner (i.e. across the skin layers) as thin as possible. The specimens are flash frozen (e.g., using dry ice or the vapor of the liquid nitrogen (without submersion) or isopentane). Without being bound by theory, it is believed that the cells are frozen without ice crystals developing in the cells. Alternatively, the fresh specimens are used without freezing. In either case, O.C.T. (Optimal Cutting Temperature) medium (contains glycerol and resin) is used to embed the specimens, and a microtome/cryostat is used to slice the specimen to the desired thickness (e.g., 8-10 micron).

FAST and SAS combo method: Using sharp scalpel for microdissection, fresh tissue samples are cut from a psoriatic lesion in small pieces. The specimens are flash frozen (e.g., using dry ice or the vapor of the liquid nitrogen (without submersion) or isopentane or dimethyl sulfoxide (DMSO)). Without being bound by theory, it is believed that the cells are frozen without ice crystals developing in the cells. The frozen specimens are smashed into smallest fragments possible. The fragments are then thawed, and each fragment is again smashed vertically and pushed horizontally. The samples are then smeared on a glass slide.

### Example 2: Identification and Characterization of Infiltrating Immune Cells in the Psoriatic Plaques or other Autoimmune Disease Target Organs

From the isolated single immune cells, clones are grown and characterized by flow cytometry to provide a full spectrum of T cells in the lesion, e.g., to elucidate the role of CD4+ T cells in psoriasis. Moreover, the following surface markers are characterized: CD8; CD4; CD3; CD25; CD127; FoxP3; CD19/20; CD45RO/RA; CD62L-; CD123; CD80/86. Serial biopsies (e.g., during partial remission and/or in flare ups) are monitored and the spectrum of T cells is characterized in the serial biopsies. CD25hi+/CD127 low, FoxP3+ population frequency and also the naïve and memory T cell population's ratios are also characterized in the serial biopsies to provide information related to dynamic changes in the autoimmune process and it self-regulatory healing process. Additionally or alternatively, genetic analysis (e.g., in depth TCR analysis), immunotyping (e.g. using cell surface markers), and/or proteomic and eicosanoid analysis (e.g., leukotrienes (LTB4 etc.)) is conducted on an immune cell.

### Example 3: Target Antigen/s Identification and Testing

T cell clones from the psoriatic lesions are used to “fish out” their corresponding autoantigens in cell culture testing. The antigen response to the T cells is characterized, along with the T cells' cytokine profiles (e.g., INF-gamma, IL-17, TNF-α, IL-6, IL-4, IL10, TGF-beta and IL-13).

In the case of psoriasis, an antigen challenge is conducted using one or more of the three putative antigens keratin, melanocytes-ADAMTSL5 and antimicrobial peptide LL37. Additional antigens are identified using the relevant patient's own skin tissue. More particularly, skin tissue is homogenized and fractionated, and fractions that induce significant T cell stimulation indices are further fractionated. The smallest responding fraction is separated by 3D gel chromatography, and the T cell clones are tested against the isolated proteins/peptides to identify antigens that induce a T cell response. Identified antigens are cloned and sequenced.

In the case of type 1 diabetes mellitus, an antigen challenge is conducted using one of more of preproinsulin, proinsulin, insulin A, insulin, chain B, insulin chain C chain, GAD65, ICA512/Ia2, HSP60, carboxypeptidase H, peripherin, ganglioside, and immunologically active fragments or variants of any of the foregoing (including post-translationally modified derivatives of any of the foregoing). Additional antigens are identified using the relevant patient's own pancreatic tissue.

In the case of rheumatoid arthritis, an antigen challenge is conducted using one of more of collagen type II, proteoglycans, human chondrocyte glycoprotein 39, heat shock proteins (HSPs), post-translationally modified proteins (such as citrullinated filaggrin), ubiquitous proteins (such as glucose-6-phosphate isomerase or p205); HSPs secreted during stress (such as BiP); N-acetylglucosamine-6-sulfatase (GNS); filamin (FLNA); and post-translationally modified variants and/or fragments of any of the foregoing. Additional antigens are identified using the relevant patient's own affected joints.

In the case of multiple sclerosis, an antigen challenge is conducted using one of more of Myelin basic protein (MBP), Myelin oligodendrocyte glycoprotein (MOG) peptides, alpha-beta-crystallin, S100beta, the DM20 isoform of proteolipid protein (PLP), myelin-associated antigen (MAG), myelin-associated oligo-dendrocyte basic protein (MOBP), 2′,3′-cyclic-nucleotide 3′-phosphodiesterase (CNPase), and post-translationally modified variants and/or fragments of any of the foregoing. Additional antigens are identified using the relevant patient's own affected cerebrospinal fluid or brain tissue.

### Example 4: Identification of the Peripheral Blood Immune Cells Response to Antigens

Peripheral blood samples are collected from subjects around the same time tissue samples (e.g., skin biopsies; joint fluid or biopsies; cerebrospinal fluid or brain biopsies; pancreatic biopsies) are collected, and findings from the tissue samples are correlated with the patient's own blood samples. More particularly, the blood samples are characterized to determine the presence or absence of T cells and/or their relevant antigens identified from the target organ (e.g., skin biopsy). Given the confirmed presence of such cells and/or antigens in the target organ, such components can be identified in blood even if they are present at low frequency.

Given the inhomogeneity associated with autoimmune disorders (i.e., the possibility of different groups of patients having different autoantigens responsive for autoimmunity), the blood test can be used for develop individualized immune modulatory therapies based on the autoantigens associated with a pathological response. The blood test can also be used as a diagnostic tool in an at-risk subject or a subject with an early stage of disease (e.g., with early or atypical disease presentation).

### Example 5: Autoimmune Cellular Biomarker in Patients with Psoriasis to Validate the Marker and Gauge the Level of Activity and/or Progression over Time of the Autoimmune Destruction

Disease-specific and/or antigen-specific memory T cells can be used to evaluate a level of autoimmune activity or disease progression. Without being bound by theory, it is believed that overall memory T cells increase because the autoimmune process activates the naive T cells, which then become disease-specific T cells and increase the memory T cell pool. Thus, the number of naive versus newly identified disease specific memory cells can be used to determine progression of disease, providing a much stronger signal with increased precision, sensitivity and specificity relative to using the entire memory cell pool in the equation, particularly earlier on in the autoimmune process. For instance, the autoimmune cellular marker for diagnosing the presence of autoimmunity disclosed in U.S. Pat. App. Pub. No. 2019/0137483 to Orban, published May 9, 2019, which is herein incorporated by reference in its entirety, is tested to determine that the marker can prognosticate the intensity of psoriasis and its impending flare-ups. More particularly, flow cytometry analysis is performed on blood samples from subjects to identify phenotypic markers. Using Fluorescence-activated cell sorting (FACS), CD4+ cells are detected using antiCD4 antibody labeled with a fluorophore. For the simultaneous detection of CD45RO, a specific antiCD45RO antibody with another fluorophore is also used. (Fluorescence-labeled antiCD4 and antiCD45RO antibodies are commercially available from various sources, such as BD Biosciences of San Jose, Calif.). Each fluorophore has a characteristic peak excitation and emission wavelength, thus making it possible to distinguish between them, e.g., by using a fluorescence-activated cell sorting instruments, such as the Becton-Dickinson FACSCALIBUR™ or FACSARIA™ system. Against the backdrop of single cell analysis, the number of naive versus identified memory cells can change over time (e.g., when cells are detected at varying time points).

### Example 6: Autoimmune Cellular Biomarker in Patients with an Autoimmune Disorder to Validate the Marker and Gauge the Level of Activity and/or Progression over Time of the Autoimmune Destruction

Disease-specific and/or antigen-specific memory T cells can be used to evaluate a level of autoimmune activity or disease progression. Without being bound by theory, it is believed that overall memory T cells increase because the autoimmune process activates the naive T cells, which then become disease-specific T cells and increase the memory T cell pool. Thus, the number of naive versus newly identified disease specific memory cells can be used to determine progression of disease, providing a much stronger signal with increased precision, sensitivity, and specificity relative to using the entire memory cell pool in the equation, particularly earlier on in the autoimmune process. For instance, the autoimmune cellular marker for diagnosing the presence of autoimmunity disclosed in U.S. Pat. Pub. App. No. 2019/0137483 to Orban, published May 9, 2019, which is herein incorporated by reference in its entirety, is tested to determine that the marker can prognosticate the intensity of an autoimmune disorder (such as rheumatoid arthritis, multiple sclerosis, or type 1 diabetes mellitus) and its impending flare-ups. More particularly, flow cytometry analysis is performed on blood samples from subjects to identify phenotypic markers. Using Fluorescence-activated cell sorting (FACS), CD4+ cells are detected using antiCD4 antibody labeled with a fluorophore. For the simultaneous detection of CD45RO, a specific antiCD45RO antibody with another fluorophore is also used. (Fluorescence-labeled antiCD4 and antiCD45RO antibodies are commercially available from various sources, such as BD Biosciences of San Jose, Calif.). Each fluorophore has a characteristic peak excitation and emission wavelength, thus making it possible to distinguish between them, e.g., by using a fluorescence-activated cell sorting instruments, such as the Becton-Dickinson FACSCALIBUR™ or FACSARIA™ system. Against the backdrop of single cell analysis, the number of naive versus identified memory cells can change over time (e.g., when cells are detected at varying time points).

### Example 7: Synthesize the Putative Antigen/s

Identified primary antigen/s are synthesized under GMP conditions. The synthesized antigens are used for preclinical testing as well as a human clinical trial. Antigens that impact the pathological immune response will be the basis of drug development. Some antigens may be more effective in particular patient populations.

### Example 8: Preclinical Animal Testing

Preclinical animal testing is conducted using any of the available animal models known for the autoimmune disease of interest. For instance, such an animal model for psoriasis is disclosed in Bochenska et al, Int. J. Mol. Sci., 2017, 18, 2514, which is herein incorporated by reference in its entirety. Two suitable animal models for candidate antigen testing are: (a) SCID/SCID mice with psoriatic skin graft and (b) Induced psoriasis in Balb/c mice using 5% imiquimod in a 62.5 mg/day dose application to the skin. In the induced psoriasis model, phenotype psoriatic plaques develop rather quickly (5-6 days on the back of the skin) and shows good similarity to the human disease itself.

Adopting toxicology protocols to test the candidate drug in the animal model, the effect of the drug on a skin lesion is evaluated with scorings used in human trials (Psoriasis Area and Severity Index (PASI) score (with PASI 90 indicating a 90% or greater improvement from baseline PASI score) and a static Physician's Global Assessment (sPGA)). The animal studies will start with doses of 2 mg human dose equivalent in the mice.

### Example 9: Human Trial

Currently immunosuppressive drugs are tested and used for psoriasis or other autoimmune conditions treatment with all the potential serious side effects. The antigen based approach described herein is non-immunosuppressive with minimal to mild potential side effects.

A Phase 1 clinical trial is conducted using the National Cancer Institute Common Terminology Criteria for Adverse Events (CTCAE) to test drug safety and monitor adverse events. The clinical trial is a placebo controlled double blinded, four-arm clinical trial with 20 patients in each arm. Three arms receive active drugs (with escalating dose) and one arm receives a placebo. The initiating dose is extrapolated from the preclinical studies. The following references are incorporated herein to the extent they enable one skilled in the art to practice methods described above:

1 Greb J. E. et al., Psoriasis. Nat Rev Dis Primers 2:16082 (2016).

2 Icen M. et al., Trends in incidence of adult-onset psoriasis over three decades: a population-based study. J Am Acad Dermatol 60(3):394-401 (2009).

3 Raphael I. et al., T cell subsets and their signature cytokines in autoimmune and inflammatory diseases. Cytokine 74(1):5-17 (2015).

4 Lande R. et al., Neutrophils activate plasmacytoid dendritic cells by releasing self-DNA-peptide complexes in systemic lupus erythematosus. Sci Transl Med 3(73): 73ra19 (2011).

5 Lande R. et al., Plasmacytoid dendritic cells sense self-DNA coupled with antimicrobial peptide. Nature 449:564-569 (2007).

6 Nederstigt C. et al., Incidence and prevalence of thyroid dysfunction in type 1 diabetes. J Diabetes Complications 30(3):420-425 (2016).

7 Bakker S. F. et al., Screening for coeliac disease in adult patients with type 1 diabetes mellitus: myths, facts and controversy. Diabetol Metab Syndr 8:51 (2016).

8 Valdimarsson H. et al., Psoriasis—as an autoimmune disease caused by molecular mimicry. Trends Immunol 30(10):494-501 (2009).

9 Prinz J. C. Melanocytes: Target Cells of an HLA-C*06:02-Restricted Autoimmune Response in Psoriasis. J Invest Dermatol 137(10):2053-2058 (2017).

10 Lande R. et al., The antimicrobial peptide LL37 is a T-cell autoantigen in psoriasis. Nature Communications 5:5621 (2014).1

11. Kim S. M. et al., Analysis of the paired TCR alpha- and beta-chains of single human T cells. PLoS ONE 7(5): e37338 (2012).

12 Bochenska K. et al., Models in the Research Process of Psoriasis. Int J Mol Sci 18(12) 2514 (2017).

13 Paul C. et al., Ixekizumab provides superior efficacy compared with ustekinumab over 52 weeks of treatment: Results from IXORA-S, a phase 3 study. J Am Acad Dermatol 80(1):70-79 (2019)

1-31. (canceled)

32. A method of preparing an immune cell sample from a subject having an autoimmune disorder, the method comprising:
obtaining a tissue sample from a tissue affected by the autoimmune disorder; and
isolating a single live immune cell in situ from the tissue sample using a technique that does not substantially alter the cellular biochemical profile of the immune cell.

33. The method of claim 32, wherein the technique comprises laser capture microdissection; micromanipulation; vacuum pulse assisted technology; immunomagnetic cell separation; density gradient centrifugation; sedimentation; adhesion; aptamers; buoyance-activated cell sorting;
fluorescence-activated cell sorting (FACS); or microfluidics.

34. The method of claim 32, wherein the autoimmune disorder is psoriasis, type 1 diabetes mellitus, rheumatoid arthritis, or multiple sclerosis.

35. The method of claim 32, wherein the tissue sample is from about 5 μm to about 15 μm thick.

36. (canceled)

37. The method of claim 32, wherein the tissue sample is obtained by blending a biopsy specimen obtained from the affected tissue in a micro blender.

38-40. (canceled)

41. The method of claim 32, wherein the method further comprises:
(a) freezing the tissue sample, and
(b) sectioning the frozen tissue sample.

42-47. (canceled)

48. The method of claim 32, wherein the tissue sample is obtained by cutting a biopsy specimen obtained from the affected tissue, and wherein the method further comprises:
(a) flash freezing the tissue sample,
(b) smashing the frozen tissue sample into fragments,
(c) thawing the fragments,
(d) smashing one or more of the thawed fragments vertically and pushing the one or more thawed fragments horizontally, and then
(e) smearing the one or more fragments on a glass slide.

49. The method of claim 32, further comprising culturing the isolated immune cell in a growth medium.

50. The method of claim 32, further comprising detecting and analysing genomics, transcriptomics, proteomics, metabolomics, T cell surface markers, TCRs, single-cell genome (DNA) sequencing, transcription factors, cytokines, newly synthesized DNA, ATP, or combinations thereof associated with the isolated immune cell.

51. (canceled)

52. The method of claim 50, wherein the detecting is performed via flow cytometry, deep sequencing, spectrophotometry, or an enzyme-linked immunosorbent assay.

53. The method of claim 32, further comprising identifying a primary antigen/s that stimulates the isolated immune cell.

54. The method of claim 53, wherein the identifying comprises culturing the isolated immune cell to generate cultured immune cells, and performing an antigen challenge on the cultured immune cells.

55. The method of claim 54, wherein performing the antigen challenge comprises contacting the cultured immune cells with an antigen and then detecting and analysing genomics, transcriptomics, proteomics, metabolomics, T cell surface markers, TCRs, single-cell genome (DNA) sequencing, newly synthesized DNA, ATP, transcription factors, cytokines, signal transducers, or combinations thereof associated with one or more of the cultured immune cells.

56. The method of claim 55, wherein the antigen is isolated from tissue obtained from the subject.

57. The method of claim 56, further comprising:
(a) homogenizing and fractionating the tissue sample,
(b) conducting the antigen challenge using the homogenized and fractionated tissue sample,
(c) detecting fractions of the fractionated tissue sample that induce T cell stimulation based on the presence or absence of T cell surface markers, transcription factors, cytokines, newly synthesized DNA, ATP, signal transducers, or combinations thereof detected during the antigen challenge,
(d) isolating polypeptides in the detected fractions by performing 3-dimensional gel chromatography,
(e) conducting a second antigen challenge using the isolated polypeptides, and
(f) detecting the isolated polypeptides that induce T cell stimulation based on the presence or absence of T cell surface markers, transcription factors, cytokines, newly synthesized DNA, ATP, signal transducers, or combinations thereof detected during the second antigen challenge.

58. The method of claim 57, further comprising detecting, in a peripheral blood sample obtained from the subject, the presence or absence of (i) one or more immune cells having T cell surface markers and/or transcription factors detected during one of the antigen challenges and/or (ii) the isolated polypeptide that induces T cell stimulation.

59. The method of claim 57, further comprising synthesizing one or more of the polypeptides that induce T cell stimulation.

60. A method of treating a subject having or at risk of having an autoimmune disorder, the method comprising administering to the subject a therapeutically effective amount of the primary antigen/s identified in claim 53.

61. The method of claim 32, further comprising culturing the immune cell in a growth medium that induces formation of regulatory immune cells.

62. A method of treating a subject having or at risk of having an autoimmune disorder, the method comprising administering to the subject a therapeutically effective amount of the regulatory immune cells induced in claim 61.
